(19) United States
(12) Patent Application Publication
(10) Pub. No.: US 2016/0030062 A1
(43) Pub. Date: Feb. 4, 2016

(54) HOLE LOCATING SYSTEM

(71) Applicant: Rich Technologies, LLC, Warsaw, IN (US)

(72) Inventor: David B Rich, Warsaw, IN (US)

(73) Assignee: RICH TECHNOLOGIES, LLC, Warsaw, IN (US)

(21) Appl. No.: 14/800,752

(22) Filed: Jul. 16, 2015

Related U.S. Application Data
(63) Continuation-in-part of application No. 14/445,773, filed on Jul. 29, 2014.

(51) Int. Cl. A61B 17/17; A61B 19/00; A61B 17/16

(52) U.S. Cl. CPC A61B 17/1703; A61B 17/1725; A61B 17/1615; A61B 17/164; A61B 19/5225; A61B 2017/0092

(57) ABSTRACT

A drill guide system is provided having a source for x-ray radiation along with a receiver to generate a visible image of the radiation. A bone with medical device or implant is located between the source and receiver. A drill with a passage transmits x-ray radiation from the source through a drill bit that then passes through the bone and implant. The source of the x-ray radiation is offset from the drilling axis and a system is used to combine multiple or varying images to reduce an occluded area generated by the drill bit. The user guides the drill bit using an image on the receiver. When the user lines up the drill with a hole in the nail, the alignment is visible on the receiver.

### CROSS REFERENCE TO RELATED APPLICATIONS

This application is a Continuation-in-Part of U.S. application Ser. No. 14/445,773 filed Jul. 29, 2014, the disclosure of which is hereby incorporated by reference.

### BACKGROUND OF THE INVENTION

This present disclosure relates to a guide system for installing and affixing an intramedullary nail into a bone. Currently there are methods and devices that are used to assist the professional during the installation of an intramedullary nail. An intramedullary nail is designed to be inserted through the center of a bone and affixed to the bone via screws that are installed through the bone. The nail has pre-existing holes along its length, but when the nail is inserted to a bone, the holes are no longer visible. One option uses a fluoroscope to sight the hole, then the user places the drill based on the image seen on a monitor. The fluoroscope is then moved out of the way, the drill is then rotated into position and drilling is started. This involves a significant amount of practice and skill, since there is no visual feedback after the fluoroscope is moved and drilling starts. One option uses magnetics to sense the holes in the nail. Another option involves a drilling template that is affixed to one end (the proximal end) of the nail. This is ineffective, since the template can become easily misaligned and the nail sometimes bends upon insertion to the bone, rendering the template useless. A bent nail or misaligned template results in an incorrectly drilled hole. An incorrectly drilled hole results in longer surgery, higher potential for infection, and other trauma that can cause post-op complications. An improved guide system is needed.

### SUMMARY OF THE INVENTION

The present disclosure describes a guide system for drilling a hole through a bone to access a hole for installing a screw in an intramedullary nail. By using a controlled beam of x-ray radiation in conjunction with a receiver, it is possible to have visual feedback on the angle and alignment of the drill to a pre-existing hole in the nail. The x-ray radiation is directed through a hollow (cannulated) drill and a receiver is placed opposite the nail. An aligned drill will show a defined shape on the receiver to guide the user in drilling the appropriately placed hole. A fiducial marker can be added either as part of the nail or separately to improve alignment accuracy. Optionally, the x-ray radiation and receiver are spaced apart from a drill at a known distance, and this distance is used to offset the hole and therefore align it to the pre-existing hole in the nail.

### BRIEF DESCRIPTION OF THE DRAWINGS

A preferred embodiment of this invention has been chosen wherein:

FIG. 1 is a side view of the system;

FIG. 2 is a partial view of the drill in FIG. 1;

FIG. 3 is a side view of the system using a cannulated drill;

FIG. 4a is a side view of the display with the hole properly aligned;

FIG. 4b is a side view of the display with the hole misaligned;

FIG. 5 is a partial view of the drill bit and bone in FIG. 1;

FIG. 6 is a section view of a drill bit showing an occluded area;

FIG. 7 is a side view of a multi-point source embodiment;

FIG. 8 is a section view of the occluded area from the embodiment in FIG. 7;

FIG. 9 is a front view of a multiple source embodiment; and

FIG. 10 is a side view of an alternate drill bit embodiment.

### DESCRIPTION OF THE PREFERRED EMBODIMENT

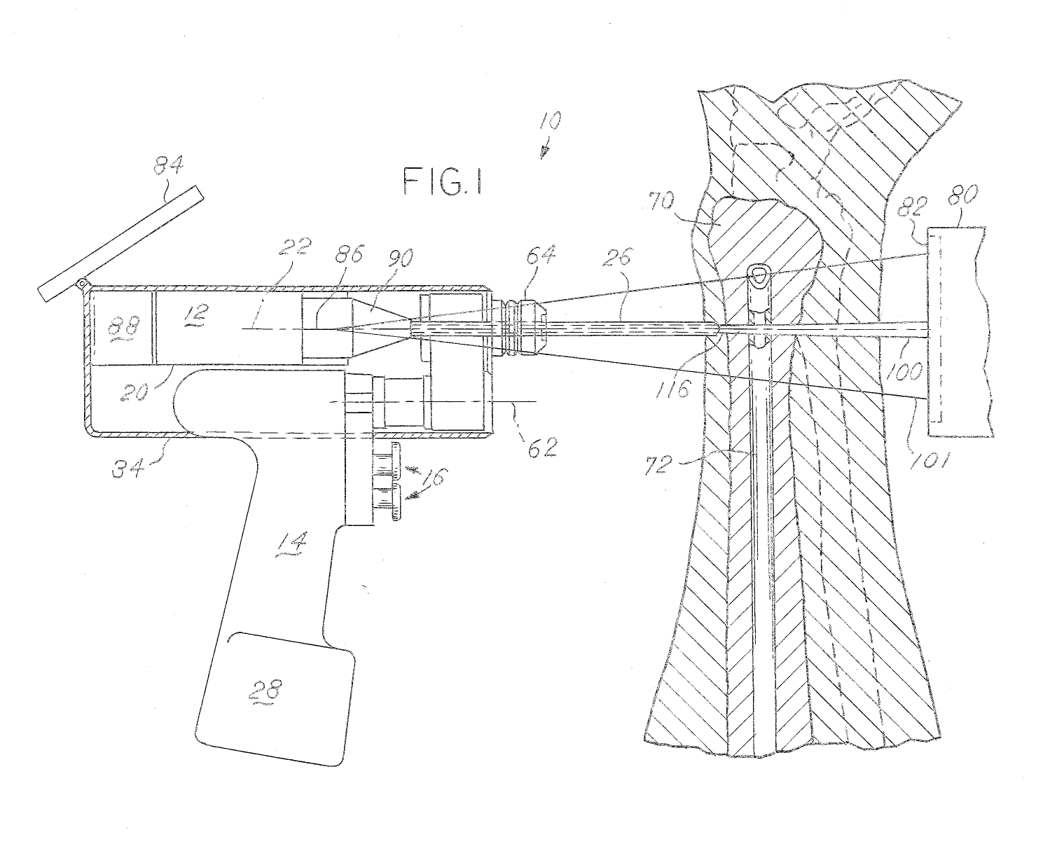

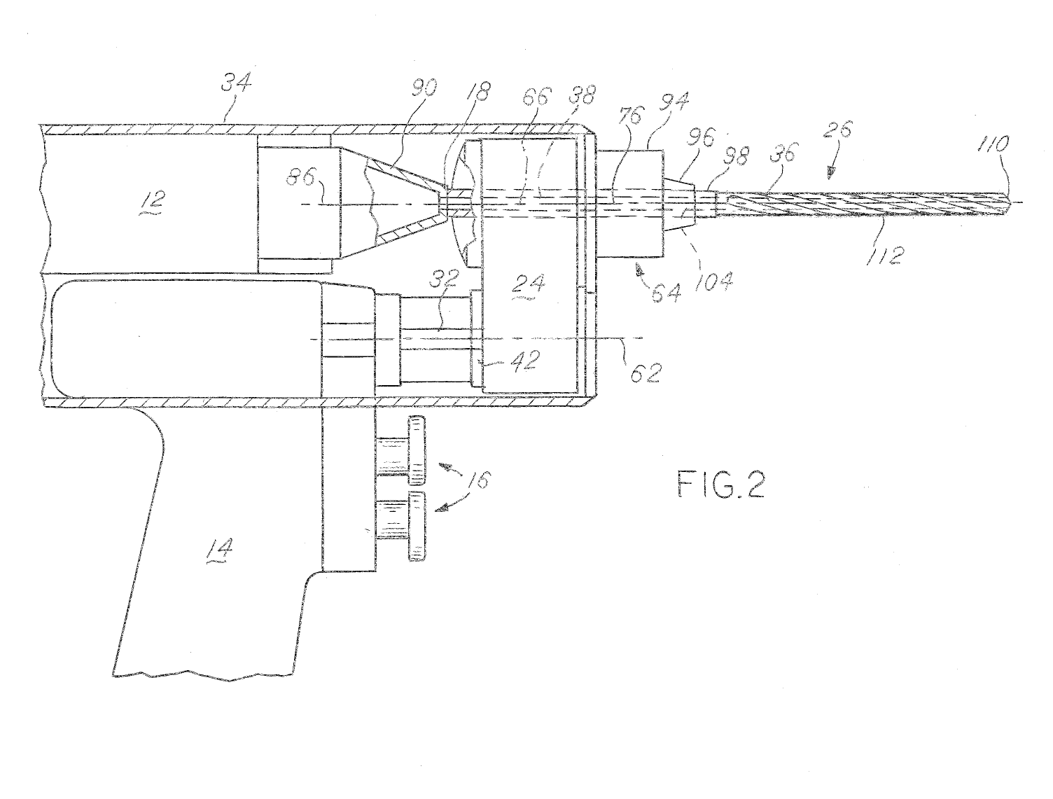

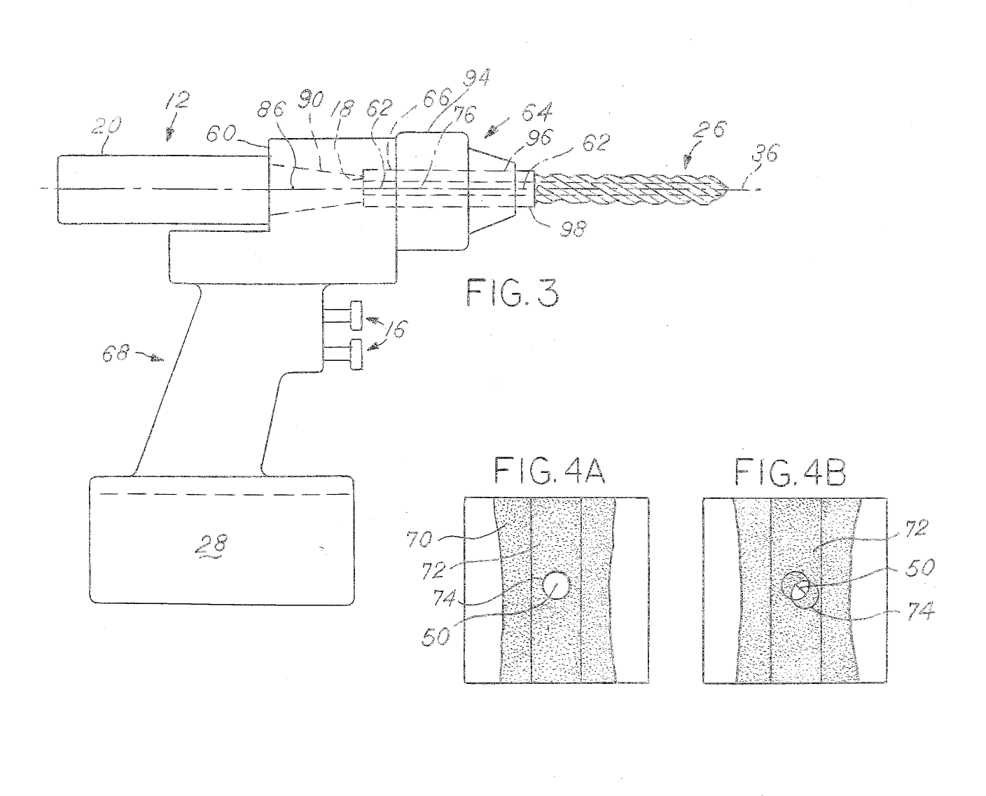

The system 10 uses an X-ray source 12 and imager 80, a drill driver 14 as shown in FIG. 1, a cannulated drill 68 as shown in FIG. 3, a collimator 24 as shown in FIG. 2, and a cannulated drill bit 26. The purpose of the system is to guide the user for drilling a hole in a visually opaque medium (such as a bone 70) but is at least partially transparent to x-ray radiation 100. Radiodensity, radiolucency, and radiopacity are all terms that describe how well a material or device blocks x-ray radiation. Radiolucency is on the more transparent side of the scale, radiopaque materials being the least transparent. Radiodense materials have properties that attenuate x-ray radiation. For the purposes of this specification, “radiation” describes x-ray radiation and “radiograph” is a representation of radiation as it is received by a medium or device and displayed such that it can be viewed by the user. The primary focus of this specification and the embodiments described involve an intramedullary nail, but the system can be used for plates or other devices that lack a useful visual alignment method. For the system to be effective, the bone, nail or other features need to show up as contrasting parts of an image on a radiograph.

In orthopedics, a broken or damaged bone 70 can be reinforced with an intramedullary nail 72 inserted through one end of the bone. Intramedullary nails have been used in the medical field for years and are well known in the art. The nail 72 can be either curved or straight but is typically round on the outside. The nail 72 is an elongate member with a proximal end and a distal end 108 as shown in FIG. 5. The nail 72 has transverse holes 74 as shown in FIG. 5 through the center 114 along the length that are angled to the center 114. The nail 72 is made from a biocompatible material, such as titanium and can be hollow, solid, or have portions that involve a combination of the two. The nail 72 as shown in FIGS. 1 and 5 is a hollow tube. In order to secure the nail 72 to the bone 70, locking screws are driven through the bone and the transverse hole 74. In order to drive the locking screw and secure the nail, a hole must first be drilled through the bone. The drilled hole 116 must line up with the axis of the transverse hole 74 for the locking screw to be properly driven into the bone 70 and nail 72. The center 114 of the outer diameter is created along the length between the proximal and distal 108 ends. The distal end 108 is inserted first and extends into a blind hole in the bone 70. The proximal end is the last portion to be inserted. After the nail 72 is completely inserted and positioned in the bone 70, a hole 116 is drilled through the side of the bone as shown in FIG. 5. For proper installation of a locking screw (not shown), the drilled hole 116 must be coaxial to the transverse hole 74 in the nail 72. The locking screw can be driven through both the drilled hole 116 and the transverse hole 74 in the nail 72. The screw anchors the bone 70 to the nail 72, thereby reinforcing or aligning it.

When a source of x-ray radiation is coupled with a device to receive and display the radiation (such as a radiograph), the user can see things that are internal to the visually opaque material. This is useful because fractures and breaks in bones are not always detectable otherwise. An x-ray source 12 is made up of a housing 20 as shown in FIGS. 1 and 3 with an aperture 18 and a generator 22 as shown in FIG. 1. The x-ray source 12 includes a power supply 88 that is integral as shown in FIG. 1 or could be external. The power supply 88 is typically a battery. A majority of the housing 20 is radiopaque, meaning it is made from materials that x-ray radiation does not penetrate through, thereby directing it only through the aperture 18 as shown in FIG. 2. Radiopaque materials (such as lead) are used to block stray radiation that would irradiate surrounding areas. The generator 22 generates x-ray radiation as a point source inside of the housing 20 and typically includes a collimator 90 to focus and direct the radiation. The collimator 90 is designed to selectively pass a narrow beam of radiation 101 from the generator 22 along an axis 86 that exits through the aperture 18, and blocks all other rays. The aperture 18 is made from radiolucent materials, meaning that x-ray radiation can pass through. As shown in FIG. 1, the radiation 101 expands outward to a certain extent after leaving the collimator 90. X-ray sources 12 with collimated radiation 101 are commonly known in the art. The x-ray source 12 can be operated continuously for a live feed of radiation or be triggered by the user to generate single snapshots. The x-ray source 12 can be made of separate components but is shown as an integrated assembly. In the separate component construction, the generator 22 is connected to the power supply 88 via high voltage cable. As shown in FIG. 1, the collimator 90 creates a central axis 86 for the x-ray radiation 100, 101.

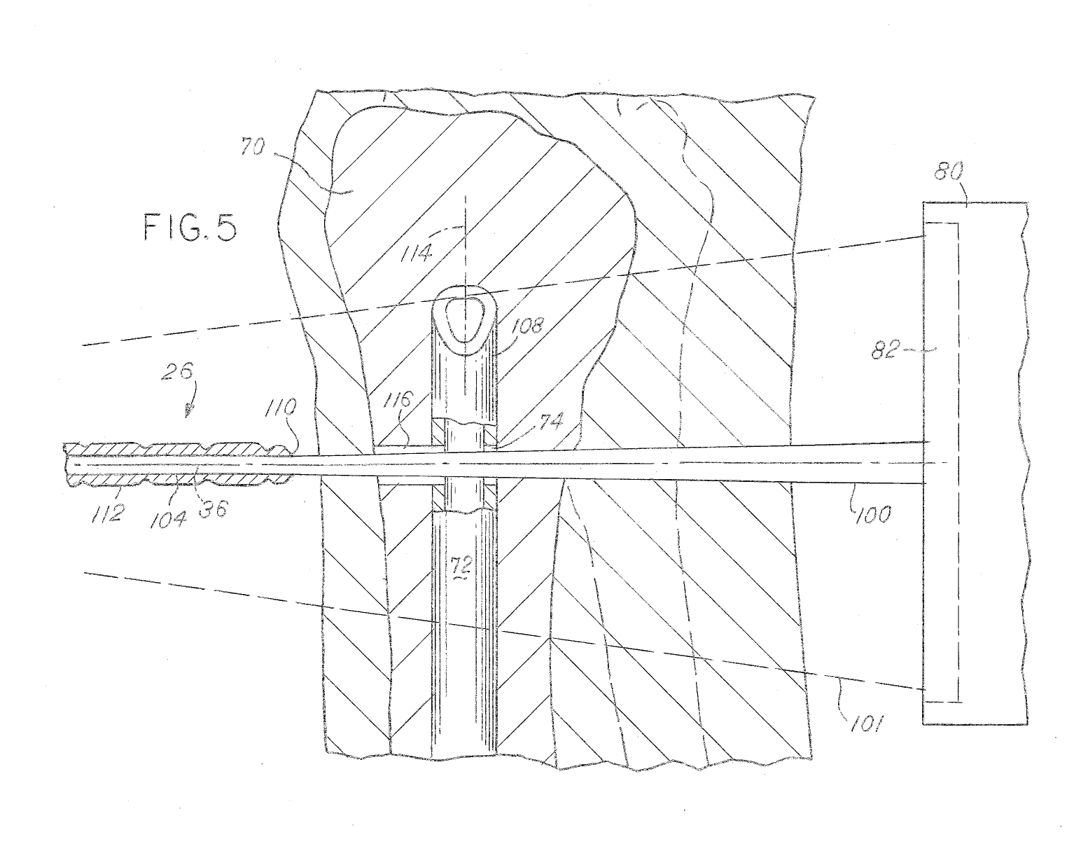

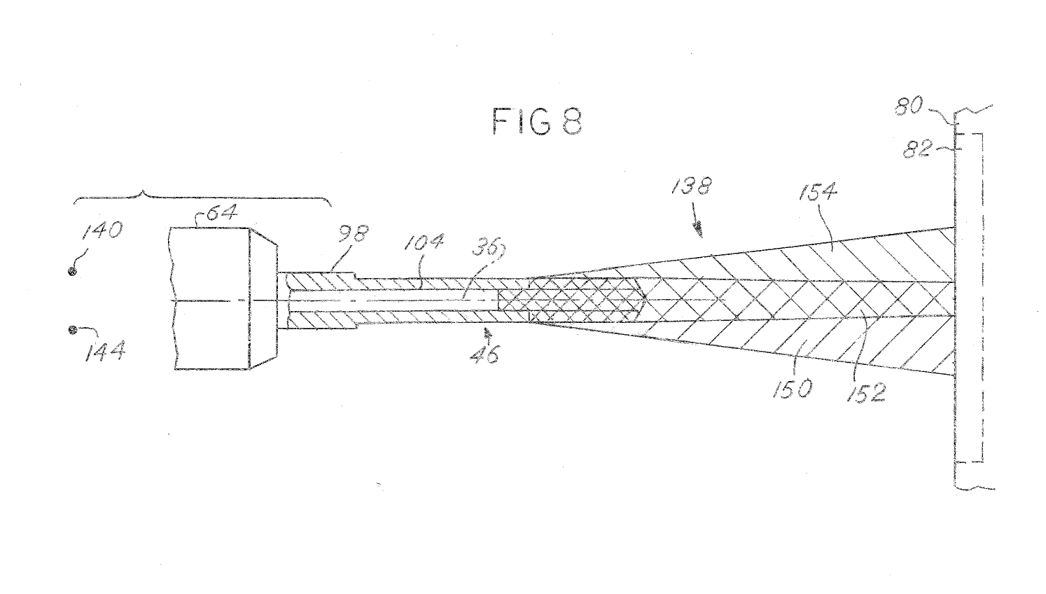

An imager 80 is made up of a panel 82 and a display 84 as shown in FIGS. 1 and 7. The panel 82 is used to receive the x-ray radiation and convert it to a radiograph image that can be viewed by the user on a display 84. X-rays penetrate various density materials in various amounts. Materials that inhibit the transmission of x-ray radiation have radiodense properties. Any physical matter with radiodense properties in the path of radiation 100, 101 (as it is projected onto the panel 82) shows up as a shadow, the intensity of the shadow is proportional to the radiodensity of the item. The different densities are visible in FIGS. 4a and 4b. For example, a lower density material (like muscle, cartilage, or other soft tissue) attenuates the radiation to a lesser extent than a more dense material. Different metals have different radiodensities and show up differently on a radiograph. The panel 82 is attached to a display 84. The display 84 can be remotely located or integrated in any part of the device 10. The display 84, as shown in FIG. 1, is mounted on the rear of the drill housing 34, source housing 20, or cannulated drill 14. Lower density materials are shown as contrasting areas as compared to more dense materials, making it possible for the user to distinguish between the two. The display 84 can be programmed to be triggered by snapshots of x-ray radiation or display a live video feed of the radiation as received by the panel 82. The snapshots limit the amount of radiation the patient receives and the live video feed gives the user a continuous feedback. Further, data processing and algorithms can be integrated into the display 84 or panel 82 such that features or conditions trigger supplemental information to be displayed. The data processing can be done in an intermediate part that is located in the path of communication between the panel 82 and the display 84. For example, transitions between a nail and a transverse hole can be highlighted with a distinct color. Fiducial markers 50 as shown in FIGS. 4A and 4B can be included with the data processing, where specific shapes could trigger highlighting, a superimposed bulls-eye, or color changes as alignment improves. Data processing and display of such data can be used as an additional tool to assist alignment by the user.

A cannulated drill 68 as shown in FIG. 3 supplies rotational torque along a driving axis 62. A cannulated drill 68 is similar to a common medical drill but has a passage 66 having a central axis 76 completely through the driving axis 62 of a cannulated chuck 64. As with any piece of medical equipment, a cannulated drill 68 is built from materials that can be sterilized and are suitable for medical use. Speed, direction, and torque are controlled by the user, typically through a control 16. As with a standard drill, the cannulated drill 68 consists of a power source (a battery pack 28 as shown in FIGS. 1 and 3), motor, gears, housing, and cannulated chuck 64. Optionally, the drill can be pneumatically driven or use a separate power source. The cannulated drill 68 is controllable remotely or locally. A cannulated chuck 64 is designed to interface or mate with an external shaft or device such as a drill bit 26. The cannulated chuck 64 is driven by the output of the gears and usually consists of a body cap 94 and jaws 96. The body cap 94 closes or opens the jaws 96 of the chuck 64 to respectively tighten or loosen around the shank 98 of a drill or another driven shaft. The body cap 94 can be tightened by hand or with a key as is commonly known in the art. While a standard medical drill driver 14 has a chuck that lacks a passage, a cannulated drill 68 has a passage 66 through the axis of the drill 62 as shown in FIG. 3. The rear of the drill is a mounting 60 and a receiving portion 30 that is used to mount the x-ray source 12. The housing 20, collimator 90 as shown in FIG. 2, or aperture 18 can be places the source 12 can mount to the drill 68. The mounting 60, inline with the chuck 64, is adapted to maintain the axis of the source 86 to the central axis 76 such that the radiation 101 can be aligned with the driving axis 62. The cannulated chuck 64 can either be made from materials with radiolucent properties or radiopaque properties. Instead of a chuck that requires a key to open and close, a quick-release chuck is possible. A quick-release chuck mates to features on a drill bit shank where torque can be transferred. Quick-release chucks are commonly known in the art.

Optionally, a standard medical drill 14 can be used in conjunction with an offset cannulated adapter 24 as shown in FIG. 2 to transmit x-ray radiation through a cannulated drill bit 26 as shown in FIG. 2. An offset cannulated adapter 24 is a device that takes x-ray radiation on one axis and input torque from another axis and combines them using a standard medical drill 14 to transmit x-ray radiation and torque for a cannulated drill bit 26. The cannulated adapter 24 is shown in FIGS. 1 and 2. The cannulated adapter 24 has housing 20, an input shaft 32, a cannulated chuck 64 and a radiolucent passage 38 through the housing giving access to the rear of the chuck. The drill as shown in FIG. 1 is a standard medical drill 14 and a cannulated adapter 24 combined with the x-ray source 12 inside of an outer housing 34 to have the same function as the cannulated drill 68 as shown in FIG. 3. Opposite the adapter 24 and coaxial to the chuck 64 is a receiving aperture to receive and align the x-ray source 12. The chuck 64 and mounting 60 have a radiolucent passage 38 about the driven axis 76 that travels through the cannulated adapter 24. The chuck 64, aperture 18, and passage 66 are coaxial. The cannulated adapter 24 has an input shaft 32 that rotates about a driving axis 62 offset from the chuck 64. A standard medical drill 14 drives the input shaft 32 through the mechanism contained in the adapter 24, torque is transferred into the chuck 64. The housing 34 or attachment 42 maintains the spatial relationship of the adapter 24 to the drill 14. The attachment 42 also prevents rotation of the adapter 24 relative to the drill 14. The adapter 24, attachment 42, and mounting allows the user to project x-ray radiation along the same axis as a drill. By attaching a cannulated drill bit 26, the drill 14 and radiation are coaxial. The cannulated adapter 24 can be made from materials with radiolucent properties or radiopaque properties, but the capability of passing some x-ray radiation from the source is necessary. If the adapter 24 is made of mostly radiopaque materials, only a portion of x-ray radiation 100 as shown in FIGS. 1 and 5 is passed from the chuck 64. If the adapter is made of mostly radiolucent materials, a larger field of radiation 101 is transmitted, in addition to the smaller field of radiation 100.

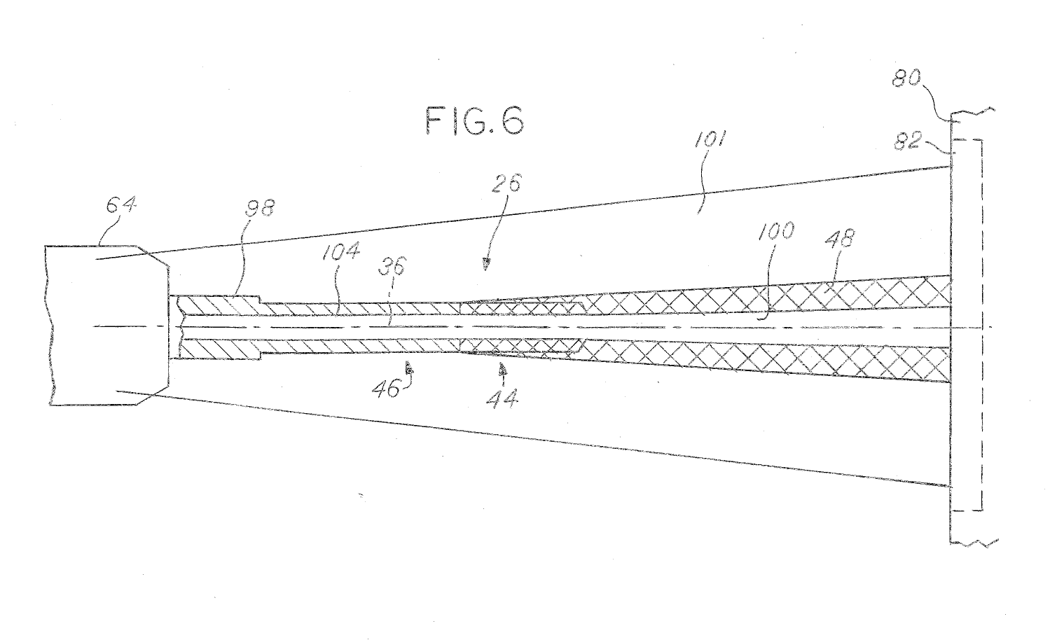

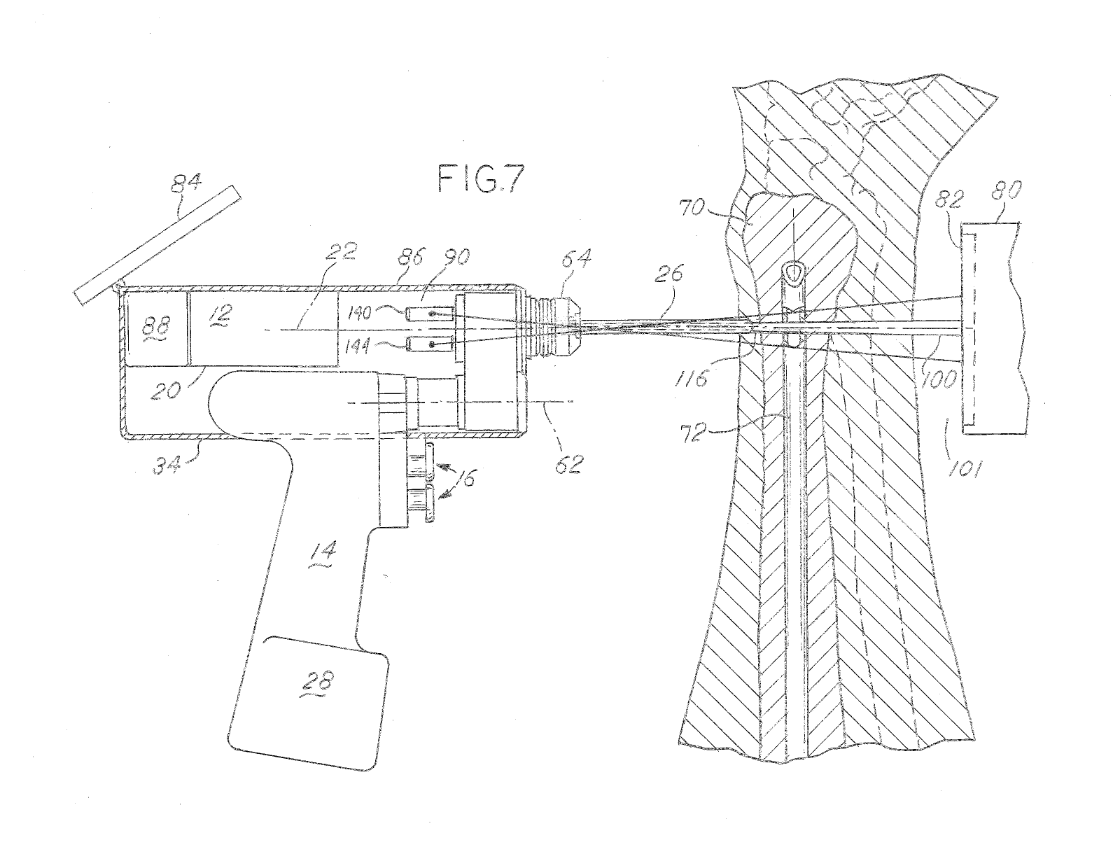

A medical cannulated drill bit 26 is a drill bit that is built similar to the standard drill bit and includes a radiolucent passage 104 FIG. 2 that can pass a portion of x-ray radiation 100 through a central axis 36 from one end to the other. The portion of x-ray radiation 100 is shown in FIGS. 1 and 5. The cannulated drill bit 26 has a shank portion 98, a tip 110, a fluted portion 112 extending from the tip towards the shank portion, and has a passage 104 about a central axis 36 that travels completely through from the shank portion 98 to the tip 110. The cannulated drill bit 26 is made from materials that can be sterilized and are suitable for medical use. Cannulated drill bits 26 are commonly known in the art. The passage 104 has radiolucent properties; it does not have to be a physical hole. As shown, the drill bit has a consistent overall diameter but many cannulated drills have a short fluted portion with the portion between the shank 98 and the tip 110 being a smooth shaft. The tip 110 is the leading edge for cutting into a material (a bone 70 FIG. 4A in particular). The fluted portion 112 is a spiral that has been cut into the outside diameter from the tip 110 towards the shank portion 98 that is designed to transport loose material away from the tip 110 as it is drilling. The shank portion 98 is designed to be held by the chuck 64. As the chuck 64 rotates, the drill bit 26 rotates along its central axis. The shank 98 can either be a smooth outer diameter or have quick-release features. FIG. 6 shows the drill bit 26 and panel 82 without a bone or nail in-between to show an occluded region 48. The cannulated drill bit 26 can be made up of radiolucent portions 46 and radiopaque or radiodense 44 portions as shown in FIG. 6. Radiodense portions 44 cast a shadow for x-ray radiation 101 creating an occluded area 48. The occluded area 48 is where a portion of the radiation 101 is obscured by the drill bit 26. The radiolucent portions 46 can exist along the length to minimize the occluded area 48. As the length of the radiodense portion 44 increases, the occluded area 48 increases.

While a standard nail 72 may have transverse holes that are used for alignment, a fiducial marker 50 as shown in FIGS. 4A and 4B can be either separately installed into the standard nail 72 before it is inserted into the bone 70 or be integral to the nail. The fiducial marker 50 can be integral to the nail for more precise alignment. The fiducial marker 50 has a different radiodensity than the nail 72 or the transverse hole 74. Typically the nail 72 is more radiodense than the transverse hole 74 or the bone 70, but does not have to be. For proper alignment, the transverse hole 74 needs to show up on a radiograph, provided the x-ray radiation is lined up with the transverse hole 74 as shown in FIGS. 4a and 4b. The fiducial marker 50 can be something as simple as a pin that is physically located coaxial to the transverse hole 74 as shown in FIGS. 4a and 4b or be made from a structure of a different shape. The fiducial marker 50 can be something that is suspended inside a material with different radiodense properties to cause a contrasting shape on the display 84. For example, if the fiducial marker 50 is an elongate pin that is suspended coaxially to the transverse hole, it would show up as a dot with a properly aligned drill bit as shown in FIG. 4a. If the drill bit 26 was misaligned, the fiducial marker would show up as a line as shown in FIG. 4b, the length shown on the display 84 thereof being directly related to the amount of misalignment. The fiducial marker 50 could take the form of two intersecting flat surfaces where the axis of intersection is central and coaxial to the transverse hole 74. An aligned drill would show a crosshair shape. Other shapes or features in the nail can have fiducial marker properties without the fiducial marker 50 being a separate piece. For example, radiolucent or radiodense features in the nail 72 can signal the user (as viewed on the display 84) as to the alignment of the drill bit 26. It is possible to use pins, grids, or tubes of various sizes or shapes to help the user dial in the alignment. The fiducial marker 50 can be integral to the transverse hole 74 and made such that when the drilled hole 116 as shown in FIGS. 1 and 5 meets the transverse hole 74, the drill bit 26 begins to displace or destroy the fiducial marker 50 and/or any supporting material around the fiducial marker. As is commonly known in the art, any material that might remain inside the body after surgery must be biocompatible.

Instead of a cannulated drill 68 or cannulated attachment 24, a drill guide could be implemented. The drill guide locates the axis of x-ray radiation to the drilling axis by a set distance. With the known distance in the guide matching a known distance between a fiducial marker in the nail 72 and the transverse hole 74, a standard drill bit can be attached to a drill driver 14. A hole 116 can be drilled by setting the guide to be offset from the transverse hole 74 by the same known distance. A drill guide gives the option of using a standard drill and drill bit. Alignment is accomplished by aligning the offset fiducial marker to the hole 116 being drilled.

In order to make the hole 116 coaxial to transverse hole 74, the x-ray source 12 is installed into the rear of the cannulated drill 68 or cannulated attachment 24 such that the central axis of the source 86 is coaxial with the central axis of passage 66. Next, a cannulated drill bit 26 is installed to the drill via the chuck 64. This arrangement makes the driving axes 62 of the drill source 86, and drill bit 36 coaxial. The assembly with a cannulated drill, as described, is shown in FIG. 3. The assembly with a standard drill and cannulated attachment is shown in FIGS. 1 and 2. The intramedullary nail 72 and bone 70 are then placed between the drill driver 14, cannulated drill 68, and the imager panel 82 as shown in FIG. 1. The source 12 , imager 80, and display 84 are enabled, making a portion of the bone with the nail visible on the display 84 as shown in FIGS. 4a and 4b. If the drill bit 26 is properly aligned, the hole 74 and/or fiducial marker 50 is visible as in FIG. 4a. If the drill bit 26 is not coaxial with the hole 74, the hole becomes visible as in FIG. 4b or not visible at all in the cases of severe misalignment. As the user begins drilling, the alignment can be monitored by watching the display 84. Corrections in the position and alignment of the drill bit 26 can be accomplished by manipulating the position or angle of the drill 14, 68. In the event a fiducial marker 50 is located inside the transverse hole, the rotating drill bit would break apart or displace the fiducial marker 50 as the drill bit penetrates the transverse hole 74.

In the event the chuck 64 is radiopaque (or is very radiodense), a portion of radiation 100 is all that passes through the radiolucent passage 104 of the drill bit. This results in an image that only shows the hole 74 when the drill bit is in sufficient proximity and alignment. In the event the chuck 64 is radiolucent (or has radiolucent properties), radiation 100 passes through the hole 104 in the drill (as shown in FIG. 1 as a portion of radiation 100) and around it 101. Radiation 100 spreads out away from the source 12 and allows the user to see a greater area on the display 84.

Drill bits 120 with radiopaque 122 and radiolucent 124 portions as shown in FIG. 10 can be used to reduce the occluded area 152 as shown in FIG. 8. Material with radiolucent properties is prone to rapid wear or breakage when used directly to drill into bone. The drill bit 120 with the radiolucent portion 124 along with a standard drill bit material used on the tip 126 can be used. Because even radiolucent materials may attenuate x-ray radiation to some extent, the drill bit 120 may have a center hole 128 to make the radiolucent portion 124 into a cannulated portion. The drill bit 120, as shown, shows where the radiopaque portion 122 has a smaller shank portion 130 that is pressed into the center hole 128. It is contemplated that the radiopaque portion 122 is connected to the radiolucent portion 124 through other means, such as a spline or metal fusing.

Alternatively, the location or modification of the x-ray source 12 is possible. As shown in FIG. 1, the generator 22 is a point, radiating outward therefrom. The point size of existing x-ray sources is small (0.5 mm to 1.0 mm) to generate a sharp image. Unlike optics, x-ray radiation does not use lenses to focus, so a small point source is necessary to generate a sharp image. It is possible to have a larger spot size (>3 mm). A larger spot size has the benefit of decreasing the occluded area 48 at the expense of image sharpness.

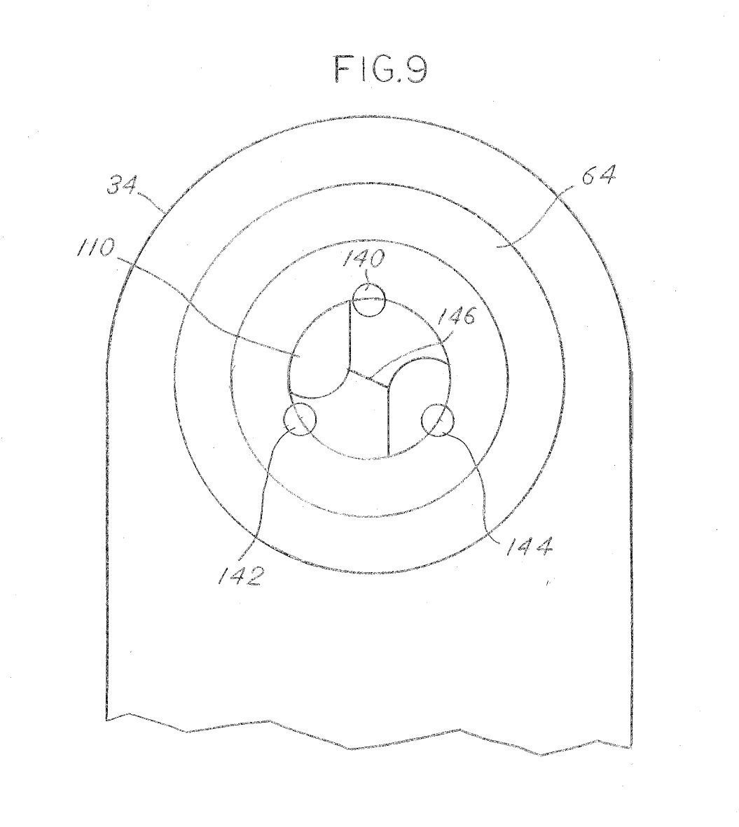

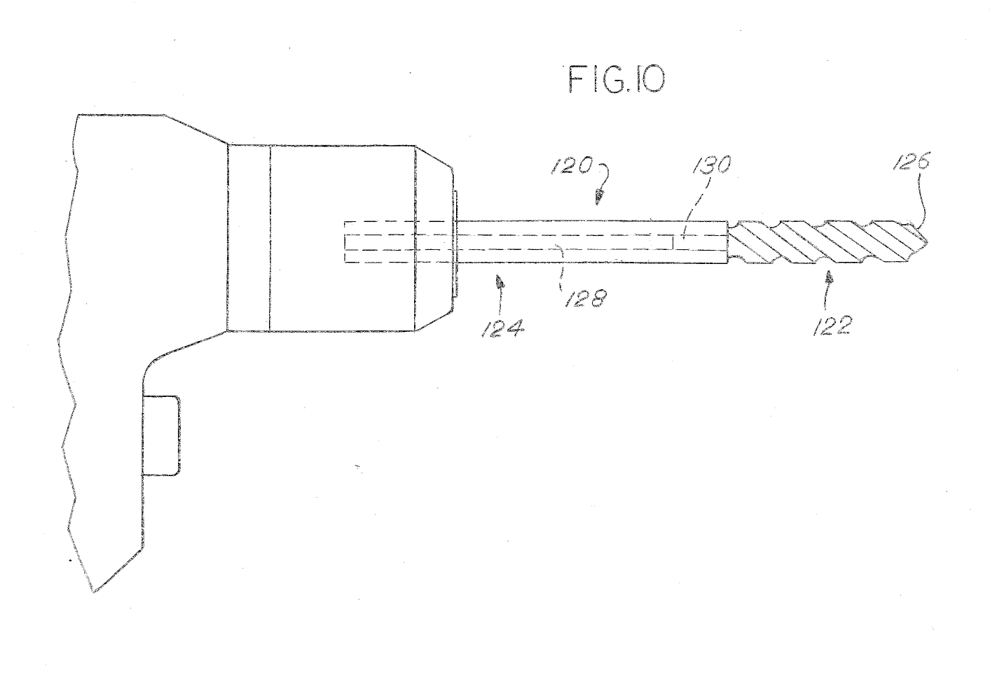

It is also possible to use multiple x-ray generators that are off axis as shown in FIGS. 7 and 9. By placing a small generator 140 away from the central axis 146 of the drill, the occluded area 138 is moved as shown in FIG. 8. By placing a multiple of generators 140, 142, 144 around the central axis 146 of the drill, it is possible to reduce or eliminate the occluded area by controlling the generators in sequence or in tandem. FIG. 9 shows a front view of the embodiment in FIG. 7. The multiple generators 140, 142, and 144 are placed around the central axis 146 at 120° increments, though other angular orientations are possible.

Further, a generator 140 can be moveable around the central axis 146 of the drill. This allows a similar result as the multiple generator embodiment, but with a single source.

When multiple generators 140, 142, 144 or a moving generator is used, the image as viewed by the user could be unintelligible because of overlap or movement. Utilizing a system to process the control, the sources, and generated images, improves the image as produced on the display 84. A control system as incorporated in the display 84 or imager 80 can enable individual generators and overlay individual images as received on the panel 82 to form a composite image. By using multiple or a moving source, the occluded area 138 can be reduced or eliminated. For example (FIG. 8), enabling the source 140 creates a first image where the image is shifted downwardly on the imager 80, creating occluded area 150. Enabling source 144 shifts the image upwardly, creating occluded area 154. The area between the two is occluded area 152. When the resulting image is processed, only occluded area 152 remains. By overlaying the two and processing the images, an image can be generated where the occluded area is significantly smaller.

An annular x-ray source can be implemented instead of multiple generators or a moving generator. The annular x-ray source would cover a similar area as the multiple generators 140, 142, and 144 but be a continuous ring of x-ray radiation instead of individual sources.

It is understood that while certain aspects of the disclosed subject matter have been shown and described, the disclosed subject matter is not limited thereto and encompasses various other embodiments and aspects. No specific limitation with respect to the specific embodiments disclosed herein is intended or should be inferred. Modifications may be made to the disclosed subject matter as set forth in the following claims.

What is claimed is:

1. A guide system for making a drilled hole in a bone having a medical device with a transverse hole, said device contacting said bone, said system comprising:
an x-ray source for producing x-ray radiation on a source axis;
an imager having a panel spaced apart from said x-ray source, said panel adapted for receiving a portion of said x-ray radiation, said imager including a display in communication with said panel, said display adapted to generate an image of said x-ray radiation as received from said panel;
a drill having a radiolucent portion between said ssxource and said panel, said radiolucent portion capable of passing radiation from said source, said drill including a rotatable chuck capable of providing rotational torque about a drill axis, said drill axis spaced from said source axis;
a drill bit having a central axis, said drill bit having a shank portion adapted to be fixed from rotation with respect to said chuck, a tip portion located at a distal end opposite said shank portion, said drill bit fixable to said chuck with said central axis coaxial to said drill axis; and
said transverse hole being visible on said display when said x-ray source is producing said x-ray radiation, said bone and said medical device are disposed between said source and said panel and said transverse hole is substantially aligned with said central axis of said drill bit.

2. The guide system of claim 1, said shank of said drill bit having a central aperture along said central axis and formed of a first material, said tip portion being formed of a second material, said first material being more radiolucent than said second material.

3. The guide system of claim 1, said guide system having at least two x-ray sources, each being offset from said drill axis.

4. The guide system of claim 3, and a controller adapted to selectively enable each of said x-ray sources to generate distinct images, and an image processor in communication with said controller, said image processor adapted to combine said distinct images from said panel to display a composite image of said transverse hole on said display.

5. The guide system of claim 4, said display affixed to said drill and in wireless communication with said panel.

6. The guide system of claim 1, said source being movable in orbit around said drill axis.

7. The guide system of claim 1, said medical device being an intramedullary nail located within said bone, said nail being an elongate member having a proximal end and a distal end and inserted into a cavity in said bone, said transverse hole being through said nail.

8. A guide system for making a drilled hole in a bone having a medical device contacting said bone and having a transverse hole therethrough, said drilled hole substantially coaxial to said transverse hole, said system comprising:
an x-ray source for producing x-ray radiation on a source axis;
an imager adapted for receiving said x-ray radiation, and said imager including a display adapted to generate a visible image of said x-ray radiation;
a drill for providing rotational torque to a chuck about a central axis, said source axis spaced from said central axis, said drill having a radiolucent portion allowing a portion of said x-ray radiation from said source through said chuck;
a drill bit having a central axis, said drill bit having a shank portion opposite a tip portion, said drill bit adapted to be fixed from rotation with respect to said chuck, said drill bit fixable to said chuck with said central axis of said chuck coaxial to said central axis of said drill bit; and
said transverse hole being visible on said display when said x-ray source is producing said x-ray radiation, said bone and said medical device are disposed between said source and said imager and said transverse hole is substantially aligned with said central axis of said drill bit.

9. The guide system of claim 8, said guide system having at least two x-ray sources being offset from said drill axis.

10. The guide system of claim 9, and a controller adapted to selectively enable each of said x-ray sources to generate separate images and an image processor in communication with said controller, said image processor adapted to combine said separate images from said panel to display a composite image of said transverse hole on said display.

11. The guide system of claim 8, said shank of said drill bit having a central aperture along said central axis and formed of a first material, said tip portion being formed of a second material, said first material being more radiolucent than said second material.

12. The guide system of claim 8, said source rotatable about said drill axis.

13. The guide system of claim 8, said medical device being an intramedullary nail located within said bone, said nail being an elongate member having a proximal end and a distal end and inserted into a cavity in said bone, said transverse hole being through said nail.

14. A guide system for locating a drilled hole in a bone, said drilled hole being drilled substantially coaxial to an existing hole in a medical device, said bone having a different radiodensity than said medical device, said system comprising:
an x-ray source for producing x-ray radiation on a source axis;
an imager located substantially opposite said x-ray source for receiving a portion of said x-ray radiation, and said imager adapted to display a visible image of said x-ray radiation as received from said source;
a drill having a chuck, said chuck capable of providing rotational torque about a drill axis, said source axis spaced from said drill axis;
a drill bit adapted to be affixed to said chuck and rotatable therewith on said drill axis, said drill bit having a central axis;
said existing hole is visible on said display when said x-ray source is producing said x-ray radiation, said bone and said device are disposed between said source and said imager and said existing hole is substantially aligned with said central axis of said drill bit.

15. The guide system of claim 14, said drill bit having an outside diameter, said source axis substantially aligned with said outside diameter when said drill bit is affixed to said chuck.

16. The guide system of claim 14, said drill having multiple x-ray sources, each of said sources substantially equally spaced from said drill axis.

17. The guide system of claim 14, said guide system having at least two x-ray sources being offset from said axis.

18. The guide system of claim 17, and a controller adapted to selectively enable each of said x-ray sources to generate a separate image and an image processor in communication with said controller, said image processor adapted to combine said separate images from said panel to display a composite image of said transverse hole on said display.

19. The guide system of claim 14, said drill having a radiolucent portion adapted to pass radiation from said source to said imager.

20. The guide system of claim 19, said medical device being an intramedullary nail located within said bone, said nail being an elongate member having a proximal end and a distal end and inserted into a cavity in said bone, said transverse hole being through said nail.
